(19) United States
(12) Patent Application Publication
(10) Pub. No.: US 2007/0042341 A1
(43) Pub. Date: Feb. 22, 2007

(54) Biodegradable polymer-ligand conjugates and their uses in isolation of cellular subpopulations and in cryopreservation, culture and transplantation of cells

(75) Inventors: Arron S. L. Xu, Apex, NC (US); Lola M. Reid, Chapel Hill, NC (US)

Correspondence Address: FOLEY AND LARDNER LLP;SUITE 500, 3000 K STREET NW, WASHINGTON, DC, 20007, US

(73) Assignee: University of North Carolina at Chapel Hill

(21) Appl. No.: 11/484,615

(22) Filed: Jul. 12, 2006

Related U.S. Application Data
(62) Division of application No. 10/931,073, filed on Sep. 1, 2004.
(60) Provisional application No. 60/499,023, filed on Sep. 2, 2003.

(51) Int. Cl. A01N 1/02; G01N 33/567; C12N 5/08

(52) U.S. Cl. 435/2; 435/7.2; 435/370

(57) ABSTRACT

The invention discloses a biodegradable particle-cell composition having at least one biodegradable particle, at least one receptive group covalently linked thereto, and a cell anchored thereto. The particle can be polylactide, a polylactide-lysine copolymer, polylactide-lysine-polyethylene glycol copolymer, starch, or collagen. The receptive group can be an antibody, a fragment of an antibody, an avidin, a streptavidin, or a biotin moiety. Moreover, the particle can also have extracellular matrix components other than collagen. The particle-cell compositions can be used for selection of cells from a population, for cell culture of anchorage-dependent cells, for cryopreservation of anchorage-dependent cells, and for transplantation as a cell therapy.

### PRIORITY CLAIM

This application is a division of U.S. application Ser. No. 10/931,073, filed Sep. 1, 2004, which claims priority to U.S. Provisional Application No. 60/499,023, filed Sep. 2, 2003, the disclosures of which are incorporated by reference herein in their entirety.

### GOVERNMENT CONTRACT RIGHTS

The U.S. Government has a paid-up license in this invention and the right in limited circumstances to require the patent owner to license others on reasonable terms as provided for by the terms of Grant No. DK09713 awarded by National Institutes of Health.

### FIELD OF THE INVENTION

The present invention relates generally to medical devices used in vivo or in vitro for production and delivery of medically useful substances. More particularly the invention relates to compositions of biodegradable natural or synthetic resins conjugated with reactive ligands. Moreover, the invention relates to methods of using such compositions for enrichment for specific subpopulations of cells, cell cryopreservation, ex vivo maintenance of cells, and cell therapy.

### BACKGROUND OF THE INVENTION

Eukaryotic cells in isolated cell culture are characteristically of two types. One type is capable of survival and proliferation in suspension culture. Among cells particularly suited for this mode of survival, are cells derived from cancers and lymphomas, and cells transformed by chemical or viral agents. In contrast, a second type of cell is that which requires anchorage to a substratum for survival and proliferation of the cells. Among cells in this latter category are adherent cells, such as those derived from solid tissues and non-transformed, adherent cell types such as those from liver, lung, brain, etc, and especially progenitor cell populations from solid tissues. Frequently, such cells require attachment to extracellular matrix components and maintenance in serum-free, hormonally defined media to grow and/or survive. The matrix component(s) can be proteins such as collagen or laminin or can be proteoglycans such as heparan sulfate proteoglycans. The composition of the hormonally defined media is unique to each cell type and to the maturational or lineage stage of the cell type; thus, progenitor cells of a given lineage have overlapping requirements with the mature cells of the lineage but they also have some requirements that are distinct. These ex vivo requirements of various adherent cell types may have been defined but even when defined are not readily scalable; that is, they can be established in routine cell cultures but are not easily used in clinical therapies, in mass cell culture, or in bioreactors that might be used clinically or industrially. Moreover, the conditions that work for storage of adherent cell types, such as cryopreservation, are impractical when the cells need to be recovered after thawing and to be used in various ways. Thus, adherent cells require unique methods for storage of the cells long-term, for separating one cell type from another, and for handling of the cells in anticipated medical uses of such cells.

Biodegradable polymers have been used for tissue engineering. Among the most extensively investigated biocompatible and biodegradable polymers used for tissue engineering, are the poly-(alpha-hydroxy acid) family of polymers and related co-polymers. Some of these polymers are approved by the F.D.A. for clinical use. Thus, they are used as the most feasible starting polymer materials in the present invention. However, the attachment of cells to such polymers remains problematic.

Compositions and methods are disclosed herein that address issues associated with anchorage-dependent cells, thereby fulfilling unmet needs relating to sorting, cell preservation, cell propagation, and medical use of cells.

### SUMMARY OF THE INVENTION

The invention provides a biodegradable polymer particle-cell composition comprising at least one biodegradable particle, at least one receptive group covalently linked thereto, and a cell anchored to said at least one receptive group. The receptive group can be any suitable group, including, but not limited to, an antibody, an antibody fragment, an avidin, a streptavidin, or a biotin moiety, a carbohydrate, a synthetic ligand, protein A, protein G, or a combination thereof. The receptive group might itself also be a ligand capable of ligand-receptor interaction.

In another aspect, the invention provides a method of cryopreservation for anchorage-dependent cells comprising allowing the cells to anchor to a composition comprising at least one biodegradable particle and freezing the mixture in the presence of suitable cryopreservatives. The cells can be provided to interact with the particles as a substantially single cell suspension.

In still another aspect, the invention provides a method of separating cells comprising providing a composition comprising at least one biodegradable polymer, at least one receptive group covalently linked thereto, at least one cell anchored to said at least one receptive group, and at least one cell not anchored to said at least one receptive group, and removing the at least one cell not anchored to the polymer. Moreover, the polymer can be fashioned into a macroparticle, microparticle or nano-particle with functional receptor groups.

In yet another aspect, the invention provides a method of cell culture of anchorage-dependent cells comprising providing a composition having at least one biodegradable polymer, at least one covalently linked receptive group, and at least one cell adherent to said at least one receptive group; and contacting this composition with cell culture medium.

In yet another embodiment, the invention provides a method of cell culture of anchorage-dependent cells comprising providing a composition having at least one biodegradable polymer, at least one covalently linked receptive group, and at least one cell adherent to said at least one receptive group; contacting this composition with cell culture medium, and wherein the cell comprises at least one of a hepatic precursor, a hemopoietic precursor, a fibroblast, a mesenchymal cell, a cardiac cell, an endothelial cell, an epithelial cell, a neuronal cell, a glial cell, an endocrine cell, or combinations thereof.

In yet still another embodiment, the invention provides a treatment of a subject in need of cell therapy, comprising administering to the subject an effective amount of a composition comprising at least one biodegradable polymer, at least one receptive group covalently linked thereto, and at least one cell anchored to said at least one receptive group. The polymer for cell therapy can be fashioned into a macroparticle, microparticle or nano-particle.

### BRIEF DESCRIPTION OF THE DRAWINGS

FIG. 1 illustrates conjugation by direct coupling with Flamine group of lysine in a protein receptor.

FIG. 2 illustrates conjugation using a polyethylene glycol residue linkage.

FIG. 3 illustrates conjugation using a biotin-streptavidin or biotin-avidin coupling.

FIG. 4 illustrates conjugation using a biotinylated polyethylene glycol linkage.

FIG. 5 illustrates conjugation using a species-specific, or secondary antibody linkage.

### DETAILED DESCRIPTION OF THE INVENTION

The present invention relates to a composition having a biodegradable polymer covalently conjugated to a receptive group or ligand. Moreover, the invention relates to this composition in further combination with a cell. The cell can be anchored to the receptive ligand or group. The receptive ligand or group can be an antibody or antibody fragment against a cell surface antigen or receptor, an avidin, a streptavidin, or a biotin moiety. The composition can further comprise one or more components of extra cellular matrix, e.g. collagen, fibronectin, laminin, or combinations thereof. The invention also relates to methods of use of such a composition for selection and isolation of populations of cells, cryopreservation of the cell particle combination, and cell culture of anchorage-dependent cells.

Definitons

Serum-free, hormonally defined medium for diploid cells (HDM-diploid cells). This medium has been found to elicit clonogenic expansion, colony formation or complete cell division of diploid subpopulations of liver parenchymal cells. This medium consist of any rich basal medium (e.g. RPMI 1640, HAM's F12) containing no copper and low calcium (<0.5 mM) and supplemented further with insulin (1-5 ug/ml), transferrin/Fe (1-10 ug/ml), and with a mixture of lipids (a mixture of free fatty acids bound to highly purified, fatty acid-free albumin; an optional but useful addition can also be high density lipoprotein at 10 ug/ml). The details of the preparation of the fatty acids is attached herewith as Appendix A.

Embryonic stromal feeders as defined herein are mesenchymal stromal feeders cells derived from embryonic tissue. The ideal for hepatic cells is stromal cells derived from embryonic liver; there is some evidence, albeit vague evidence, for tissue-specificifity. The inventors have defined the age limit in rats but not in humans (e.g. the embryonic stroma are obtained ideally from embryonic rat livers from gestational ages E13-EB17). In humans, we can make only guesses as to the corresponding gestational ages such as human embryonic livers from week 12-18 of gestation. There is no data from this lab to confirm that speculation. However, most importantly these feeder cells are age-specific, and the most active forms are from embryonic tissue. One can use “STO” cells, embryonic stromal cell line derived from mouse embryos and used routinely for maintenance of embryonic stem cells (ES cells). The STO cells do not give quite the same effect as embryonic liver stroma but do well enough that investigators use them to avoid having to prepare primary cultures of embryonic tissues.

Clonogenic expansion as defined herein refers to cells that can be subcultured and expanded repeatedly even at very low seeding densities (ultimately 1 cell/dish).

Colony formation involves the formation of a colony of cells from the seeded cells but involves a limited number of divisions (typically 5-7 cell divisions) over a relatively short period of time (1-2 weeks). The cells cannot be subcultured easily if at all. Unlike clonal expansion, colony formation may incorporate differentiation steps that preclude indefinite cell division and subculture.

Primitive hepatic stem cells as defined herein are pluripotent cells with clonogenic expansion potential and with co-expression of cytokeratin 19 (CK19) and albumin (i.e. biliary and hepatocytic markers, respectively) but an absence of expression of alpha-fetoprotein. In human liver lineages from fetal livers, these cells also co-express N-CAM, Epithelial CAM (EP-CAM), and CD 133 and will clonogenically expand on tissue culture plastic and in HDM-diploid cells.

Proximal hepatic stem cells (also called hepatoblasts) as defined herein are pluripotent cells with clonogenic expansion potential and with co-expression of cytokeratin 19 (CK19), albumin, and alpha-fetoprotein. Inhuman liver lineages from fetal livers, these cells also co-express I-CAM, Epithelial CAM (Ep-CAM) and CD133 and will clonogenically expand on embryonic stromal feeders (e.g. STO cells) and in HDM-diploid cells.

Committed Progenitors as defined herein are unipotent progenitors that can give rise to either hepatocytes (committed hepatocytic progenitors) or biliary epithelial cells (committed biliary progenitors). These cells will form colonies on embryonic stromal feeders and in HDM-diploid cells. It is unclear yet if they can clonogenically expand under these or other other conditions.

Diploid Adult Hepatocytes (also called “small hepatocytes”) as defined herein are diploid hepatocytes that range in size from 15-20 um, that express various adult-specific functions (e.g. PEPCK, glycogen), do not express EP-CAM, CD133, or N-CAM, and will form colonies under various conditions but do so ideally if plated on embryonic stromal feeders and in HDM-diploid cells but further supplemented with epidermal growth factor (EGF) at 10-50 ng/ml.

Polyploid hepatocytes as defined herein are hepatocytes that are polyploid (can range from tetraploid or 4N up to 32N depending on the mammalian species). These are the mature cells of the liver and have been found to undergo DNA synthesis but with limited, if any, cytokinesis under regenerative conditions.

Progenitors as defined herein is a broad term comprising all subpopulations of stem cells and committed progenitors.

Precursors as defined herein is a functional term indicating that a specific subpopulation of cells is a precursor to another subpopulation of cells. For example, the primitive hepatic stem cells are precursors to the hepatoblasts; the hepatoblasts are precursors to the committed progenitors; the diploid adult hepatocytes are precursors to the polyploid hepatocytes.

As used herein, the term “cryopreservation” relates to the freezing of cells and/or tissues under conditions that maintain the cells' viability upon subsequent thawing. General techniques for cryopreservation of cells are well-known in the art; see, e.g., Doyle et al., (eds.), 1995, Cell & Tissue Culture: Laboratory Procedures, John Wiley & Sons, Chichester; and Ho and Wang (eds.), 1991, Animal Cell Bioreactors, Butterworth-Heinemann, Boston, which are incorporated herein by reference.

The biodegradable polymer-ligand conjugates of the invention are termed cell-receptive particles, or more simply particles. These terms are used with all embodiments of the biodegradable polymer-ligand conjugates including, but not limited to, direct antibody conjugates, conjugates to fragments of antibodies, avidin conjugates, biotin conjugates, fibronectin conjugates, conjugates biodegradable particles and antibody with long spacer linkers, such as, but not limited to, PEG linkers and anti-antibody conjugates.

Preparation of Polymers

Several kinds of biocompatible and biodegradable polymers are suitable for use in the current invention, including, but not limited to, polylactide, polylactide-lysine copolymer, polylactide-lysine-polyethylene glycol copolymer, starch, alginate and proteins. Suitable proteins are collagen, gelatin, poly-lysine, laminin, fibronectin, or combinations thereof. One embodiment of the invention uses the poly-(alpha-hydroxy acid)-lysine copolymers, and/or poly(lactide-co-glycolide, PLGA) copolymer. PLGA can be activated by coupling reagent such as, but not limited to, glutaraldehyde prior to coupling with amino containing ligands or proteins (Seifert, Romaniuk and Groth, 1997 Biomaterials 18: 1495-1502). Biodegradable PLGA polymers may also be coupled with amino groups of protein A or protein G, or other protein receptors by bifunctional linker such as (3 [(2-aminoethyl) dithio] propionic acid, AEDP) that is a commercially available linker. In the present invention, the poly-(alpha-hydroxy acid) family of polymers and copolymers are also used to prepare biocompatible and biodegradable beads without surface reactive groups, thus providing the a core structure of degradable polymer particles.

As used herein, a polymer, or polymeric matrix, is “biocompatible” if the polymer, and any degradation products of the polymer, are substantially non-toxic to the recipient and also present no significant deleterious or untoward effects on the recipient's body, such as a significant immunological reaction at the injection site.

As used herein, “biodegradable” means the composition will degrade or erode in vivo to form smaller chemical species. Degradation can result, for example, by enzymatic, chemical and/or physical processes. Suitable biocompatible, biodegradable polymers include, for example, and not by way of limitation, poly(lactides), poly(glycolides), poly(lactide-co-glycolides), poly(lactic acid)s, poly(glycolic acid)s, poly(lactic acid-co-glycolic acid)s, polycaprolactone, polycarbonates, poly(amino acids), polyorthoesters, polyetheresters, copolymers of polyethylene glycol and polyorthoester, blends and copolymers thereof.

For example, and not by way of limitation, biocompatible, non-biodegradable polymers suitable for use in the present invention include non-biodegradable polymers selected from the group consisting of polyacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, blends and copolymers thereof.

[Further, the terminal functionalities of a polymer can be modified. For example, polyesters can be blocked, unblocked or a blend of blocked and unblocked polyesters. A blocked polyester is as classically defined in the art, specifically having blocked carboxyl end groups. Generally, the blocking group is derived from the initiator of the polymerization and is typically an alkyl group. An unblocked polyester is as classically defined in the art, specifically having free carboxyl end groups.

Acceptable molecular weights for polymers used in the present invention can be determined by a person of ordinary skill in the art taking into consideration factors such as the desired polymer degradation rate, physical properties such as mechanical strength, and rate of dissolution of polymer in solvent. Typically, an acceptable range of molecular weights is of about 2,000 Daltons to about 2,000,000 Daltons. In a preferred embodiment, the polymer is a biodegradable polymer or copolymer. In a more preferred embodiment, the polymer is a poly(lactide-co-glycolide) (hereinafter “PLGA”) or derivatives with a lactide:glycolide ratio of about, but not limited to, 1:1 and a molecular weight of about 5,000 Daltons to about 70,000 Daltons. In an even more preferred embodiment, the molecular weight of the PLGA used in the present invention has a molecular weight of about 5,000 Daltons to about 42,000 Daltons.

In one embodiment, copolymers containing amino acids with reactive side chains, such as lysine, are co-polymerized with lactic acid containing monomer, the glycolic acid-containing monomer, or any other monomer with a similar mechanism of polymerization. As examples, the lactic acid containing monomer can be a lactide and the glycolic acid containing monomer can be a glycolide. The reactive sites on the amino acids are protected with standard protecting groups. Similarly, the polymer with protected side groups can be deprotected to generate reactive amino groups. The de-protected poly(lactic) acid-lysine copolymer can be further covalently coupled with receptive agents by conjugating the epsilon amino group of lysine residues to form direct tethered conjugates after fabrication of the poly(lactic) acid-lysine copolymer into desirable porous particles. In some embodiments the receptive group can be a protein including, but not limited to, an antibody, antibody fragment, collagen, laminin, fibronectin, avidin or streptavidin, or a small molecule ligand group including, but not limited to, biotin and RGD-containing peptides, protein A or protein G.

As used herein, the antibodies contemplated for use in the present invention include, but are not limited to polyclonal antibodies, monoclonal antibodies (mAbs), humanized or chimeric antibodies, single chain antibodies, Fab fragments, F(ab′).sub.2 fragments, fragments produced by a Fab expression library, anti-idiotypic (anti-Id) antibodies, and epitope-binding fragments of any of the above.

As used herein, a small molecules ligand group is one having a molecular weight of no greater than 10,000 dalton, more preferably less than 5,000 dalton. For example, combinatorial technologies can be employed to construct combinatorial libraries of small organic molecules or small peptides. See generally, e.g., Kenan et al., Trends Biochem. Sc., 19:57-64 (1994); Gallop et al., J. Med. Chem., 37:1233-1251 (1994); Gordon et al., J. Med. Chem., 37:1385-1401 (1994); Ecker et al., Biotechnology, 13:351-360 (1995). Such combinatorial libraries of compounds can be used as the receptive group in the present invention. Random peptides can be provided in, e.g., recombinantly expressed libraries (e.g., phage display libraries), or in vitro translation-based libraries (e.g., mRNA display libraries, see Wilson et al., Proc Natl Acad Sci 98:3750-3755 (2001)). Small molecule ligands also include those mocules such as carbohydrates, and compounds such as those disclosed in U.S. Pat. No. 5,792,783 (small molecule ligands are defined herein as organic molecules with a molecular weight of about 1000 daltons or less, which serve as ligands for a vascular target or vascular cell marker), peptides selected by phage-display techniques such as those described in U.S. Pat. No. 5,403,484, and peptides designed de novo to be complementary to tumor-expressed receptors; antigenic determinants; or other receptor targeting groups.

As used herein, the term “RGD” refers not only to the peptide sequence Arg-Gly-Asp, it refers generically to the class of minimal or core peptide sequences that mediate specific interaction with integrins. Thus, an “RDG targeting sequence” encompasses the entire genus of integrin-binding domains. Directing a molecule to the surface of the cell is known to facilitate uptake of the molecule, presumably through endocytic means. See, for example, Hart et al., J. Biol. Chem. 269:12468-74 (1994) (internalisation of phage bearing RGD); Goldman et al, Gene Ther. 3:811-18 (1996) (RGD-mediated adenoviral infection) and Hart et al., Gene Ther. 4:1225-30 (1997) (RGD-mediated transfection). Thus, a targeting domain in many cases will act as an internalization domain, as well. Many such targeting signals are known in the art. One class of targeting signals, which bind specifically to integrins (points of extracellular matrix attachment), bears a the peptide signal sequence based on Arg-Gly-Asp (RGD). Yet another class includes peptides having a core of Ile-Lys-Val-Ala-Val (IKVAV). See Weeks et al., Cell Inmunol. 153:94-104 (1994).

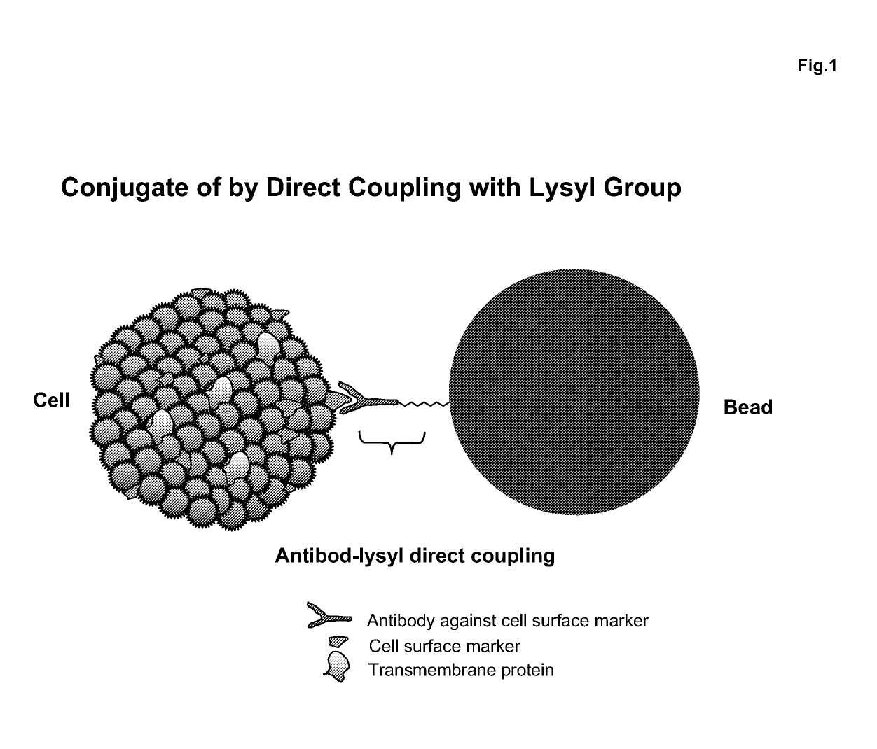

FIG. 1 refers to the hydrophilic nature of the lysine linkage that allows the coupling reaction to proceed in an aqueous medium.

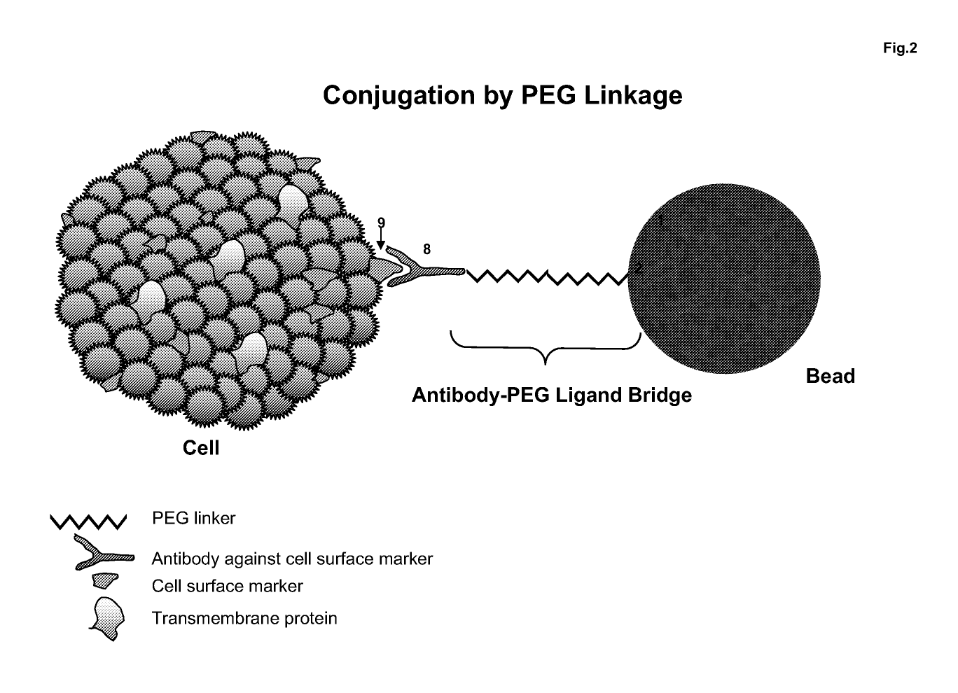

As depicted in FIG. 2, to extend further the capacity of the co-polymer in tethering proteins (including, for example, antibodies), polyethylene glycol (“PEG”) linkers can be activated by sulfonyl chloride and analogs, and coupled to the primary amine groups, such as, but not limited to, epsilon-amino group of lysyl residues or a protein, thus forming an extended linkage with three-dimensional distribution and structural characteristics. Linker structures of various lengths and linearities that are commercially available, are suitable for the invention, so that a variety of surface distributions are obtainable. A variety of linkers, such as, without limitation, those commercially available from, Pierce Chemical Co. are suitable for use in the methods of the present invention. Alternatively, such linker structures may be synthesized using routine synthetic organic chemistry methods available to those of skill in the art. The surface distribution of receptive sites is an important property affecting the density and distribution of the cell-targeting receptor molecules on the surface of the novel polymers. In any event, the surface distribution of receptive cluster sites adopted must be sufficient to enable cell contacts that is important to cell growth and differentiation, mobility and morphology (e.g., Cima, L. G 1994, J. Cellular Biochemistry 56:155-161). The surface distribution of receptive sites can be routinely determined on a case by case basis for the specific cell type being harvested using specific assays available to those of skill in the art. Such characterizations include, without limitation, determining the binding of radioactively or fluorescently labeled receptors targeted by ligands on polymer surface (e.g, Rolwey J. A., Madlambayan, G., Mooney, D. J. 1999, Biomaterials 20:45-53; Massia, S. P., Hubbell, J. A. 1991, J. Cell Biology 114:1089- 1100), X-ray and neutron reflectivity analysis (e.g., Russell, T. P. 1990 Material Science Reports 5:171-271), and binding analysis of immunofluorescence labeled antibodies of surface receptive groups (e.g., Massia, S. P., Hubbell, J. A. 1991, J. Cell Biology 114:1089-1100. As illustrated in FIG. 2, depending on the structure of the linkers, the end copolymers can have linear or branched linkers with single or multiple reactive groups. The linkers are preferentially hydrophilic, and can be exposed to aqueous medium, thus becoming accessible to incoming coupling agents.

Fabrication of Novel Polymers Into Scaffold or Beads

Another important aspect of the present invention relates to the fabrication of the biodegradable polymers into particles, beads, fiber, or scaffolds. Porous particles of a size up to about 1000 micrometers (microns) can be prepared with the method of the present invention. Moreover, the invention discloses ways of modifying the surface porosity, the internal porosity of the particles, the degradation, and the distribution of surface reactive groups. Polymer particles larger than about 500 microns in diameter, termed macroparticles, are prepared by a low temperature rapid freezing of polymer droplets embedded with NaCl or similar crystal particles of a defined size. The polymer particles may have size ranges including, but not limited to, about 500 microns, about 550 microns, about 600 microns, about 650 microns, about 700 microns, about 750 microns, about 800 microns, about 850 microns, about 900 microns, about 950 microns, about 1000 microns, about 1050 microns, about 1,100 microns, or larger as the need may arise. This method creates a porous structure upon leaching of the embedded crystals by a solvent chosen for dissolution of the crystal but not the polymer.

For fabrication of particles of a size from about 200 to about 500 microns, termed microparticles, an emulsion of a polymer of a defined formulation is dispersed as fine droplets into aqueous media in the presence of a surfactant. Continued dispersion of the droplets allows the extraction and evaporation of the solvent, leaving the polymer particles solidified. The polymer microparticles may have size ranges including, but not limited to, about 200 microns, about 250 microns, about 300 microns, about 350 microns, about 400 microns, about 450 microns, about 450 microns, about 500 microns, etc. Small polymer particles less than about 200 microns in diameter, termed nanoparticles, are prepared by rapidly dispersing polymer solution into fine droplets using ultrasonic shear forces typically delivered by an ultrasonic atomizer.

The polymer of the small particles solidifies that low temperatures and the solvent for the polymer is removed by a second or third solvent. The polymer microparticles may have size ranges including, but not limited to, about 25 microns, about 50 microns, about 75 microns, about 100 microns, about 125 microns, about 150 microns, about 175 microns, about 200 microns, etc. Thus, the particle can be macroparticle, microparticle, nanoparticle, or any combination thereof. The polymer can also be formed into fibers, including hollow fibers.

Direct Coupling of Antibody and Other Proteins Onto Polylactice Acid (-Lysine Copolymer)

Proteins of interest can be conjugated to biodegradable polymer particles or scaffold using cross-linking reagents. Among the suitable proteins, but without limitation, are antibodies, avidin, streptavidin, and extracellular matrix proteins, peptides containing RGD sequence, protein A/G.

Antibodies targeting cell surface markers and other proteins can be directly conjugated with epsilon amino groups of lysyl residues of the copolymer present on the polymer bead surface thereby forming an antibody or other protein tethered to the surface. A variety of coupling reagents, e.g., glutaraldehyde, but not limited to, that are commercially available (e.g., from Pierce Chemical Co) can be used to couple the antibody or other protein to the biodegradable polymer. For example, 1-ethyl-3-(3-dimethylaminopropyl) carbodiimide hydrochloride can be reacted with buffer in the pH range 4-6 in the presence of the antibody, or other protein, and the particles. The tethering can also occur in general as a two-step process using 6-(4-azido-2-nitrophenylamino) hexanoic acid N-hydroxy succinimide ester. In this method, the particle is initially reacted in the dark with the succinimide reagent, at a pH range of 6.5 to 8.5. Subsequently antibody or other protein is added and coupling is initiated by irradiation at 250-350 nanometers to produce a reactive nitrene. The nitrene inserts into nearby molecules, including the antibody. Unreacted reagents can subsequently be removed by washing with aqueous medium.

A number of other reagents that cross-link primary amine groups are equally suitable for tethering antibody or other protein to biodegradable particles, including: S-acetylmercaptosuccinic anhydride; S-acetylthioglycolic acid N-hydroxy- succinimide ester; 4-azidobenzoic acid N-hydroxy succinimide ester; N-(5-azido-2-nitrobenzoyloxy) succinimide; bromoacetic acid N-hydroxysuccinimide ester; dimethyl 3,3′- dithio-bis(propionimidate) dihydrochloride; dimethyl pimelimidate dihydrochloride; dimethyl suberimidate dihydrochloride; 4,4′, dithio-bis(phenyl azide); 3,3′, dithio-bis(propionic acid) N-(hydroxysuccinimide ester); ethylene glycol-bis(succinic acid N-hydroxy succinimide ester); 6-(iodoacetamido) caproic acid N-hydroxysuccinimide ester; iodoacetic acid N-hydroxy succinimide ester; 3-maleimidobenzoic acid N-hydroxysuccinimide ester; gamma-maleimidobutyric acid N-hydroxy succinimide ester; epsilon maleimidocaproic acid N-hydroxysuccinimide ester; 4-(N-maleimidomethyl) cyclohexane- 1-carboxylic acid N-hydroxy succinimide ester; 4-(N-maleimidomethyl) cyclohexane-l-carboxylic acid 3-sulfo-N-succinimide ester sodium salt; beta maleimidopropionic acid N-hydroxysuccinimide ester; bis(polyoxyethylenebis[imidazoyl carbonyl]); 3-(2-pyridyldithio)propionic acid N-hydroxysuccinimide ester; suberic acid bis(N-hydroxy succinimide ester); and bis(sulfosuccinimidyl) suberate.

The coupling of antibody or other protein to biodegradable particles can occur at various concentrations of cross-linker from about 10⁻⁹ to about 10⁻³M. In one embodiment, the concentration of about 10⁻⁵M is used.

The antibody concentration can be between about 20 ng/ml and about 20 mg/ml. The other protein concentration can be between about 5mg/ml and about 50 mg/ml. In one embodiment, the antibody or other protein concentration for the coupling reaction is about 2 mg/ml. The particle concentration can be between about 10⁻¹ and about 10⁻²M lysine equivalents. In one embodiment, the concentration of particles is about 10⁻³M lysine equivalents.

The surface distribution, the length of the tether and the optimization of the interaction between antibodies, or other proteins, and cell surface markers can be modified by those skilled in the art using, for example, polyethylene glycol (PEG) linkers for coupling the biodegradable polymer to the antibody. One such polyethylene glycol linker is described above as bis(poly-oxyethylene bis[imidazoyl carbonyl]). The specificity of the tethered antibodies primarily determines the cell selectivity of the antibody-polymer conjugates. Fragments of antibodies, for example Fab or Fab fragments, including Fab, are suitable for tethering to the biodegradable polymer.

Monoclonal antibodies for use in the methods of the present invention can be obtained by any technique which provides for the production of antibody molecules by continuous cell lines in culture. These include, but are not limited to the hybridoma technique of Kohler and Milstein, (Nature, 256:495-497, 1975; and U.S. Pat. No. 4,376,110), the human B-cell hybridoma technique (Kosbor et al., Immunology Today, 4:72, 1983; Cole et al., Proc. Natl. Acad. Sci. USA, 80:2026-2030, 1983), and the BV-hybridoma technique (Cole et al., Monoclonal Antibodies And Cancer Therapy (Alan R. Liss, Inc. 1985), pp. 77-96. Such antibodies can be of any immunoglobulin class including IgG, IgM, IgE, IgA, IgD and any subclass thereof. The hybridoma producing the mAb of this invention can be cultivated in vitro or in vivo. Production of high titers of mAbs in vivo makes this the presently preferred method of production.

In addition to the use of monoclonal antibodies in the method of the present invention, chimeric antibodies and single chain antibodies may also be used. A chimeric antibody is a molecule in which different portions are derived from different animal species, such as those having a variable region derived from a murine mAb and a constant region derived from human immunoglobulin. “Chimeric antibodies” can be made by splicing the genes from a mouse antibody molecule of appropriate antigen specificity together with genes from a human antibody molecule of appropriate biological activity (see, Morrison et al., Proc. Natl. Acad. Sci., 81:6851-6855, 1984; Neuberger et al., Nature, 312:604-608, 1984; Takeda et al., Nature, 314:452-454, 1985; and U.S. Pat. No. 4,816,567).

Alternatively, techniques described for the production of single chain antibodies (e.g., U.S. Pat. No. 4,946,778; Bird, Science, 242:423-426, 1988; Huston et al., Proc. Natl. Acad. Sci. USA, 85:5879-5883, 1988; and Ward et al., Nature, 334:544-546, 1989), and for making humanized monoclonal antibodies (U.S. Pat. No. 5,225,539), can be used to produce single chain antibodies for use in the methods of the present invention.

In one embodiment, the particles are coated with growth-permissive, natural extra-cellular matrix (“ECM”) and cross-linked to form a matrix surface for anchorage of cells to the matrix. Thus, these ECM-coated particles provide an attachment support for anchorage-dependent cells. The above cross-linkers are used to attach the ECM to the particles using methods standard in the art. The ECM can include any of the variants of collagen, fibronectin, laminin, or combinations thereof.

In another embodiment, avidin or streptavidin are conjugated to the biodegradable particles by cross-linking with cross-linkers using methods standard in the art.

The polymer molecules can be cross-linked to protein in any manner suitable to form an active conjugate according to the present invention. For example, biodegradable polymers can be cross-linked using bi- or poly-functional cross-linking agents which covalently attach to two or more polymer and protein molecules. Exemplary bifunctional cross-linking agents include derivatives of aldehydes, epoxies, succinimides, carbodiimides, maleimides, azides, carbonates, isocyanates, divinyl sulfone, alcohols, amines, imidates, anhydrides, halides, silanes, diazoacetate, aziridines, and the like. Alternatively, cross-linking may be achieved by using oxidizers and other agents, such as periodates, which activate side-chains or moieties on the polymer so that they may react with other side-chains or moieties to form the cross-linking bonds. An additional method of cross-linking comprises exposing the polymers and protein to radiation, such as gamma radiation, to activate the side polymer to permit cross-linking reactions.

Conjugates can be formed between biodegradable particles and proteins including, but not limited to, polyclonal antibodies, monoclonal antibodies, chimeric antibodies or fragments thereof, collagen I, collagen III, collagen IV, laminin, fibronectin, avidin, and streptavidin.

Biotinylation of Reactive Groups On Surfaces Of Polymer Beads

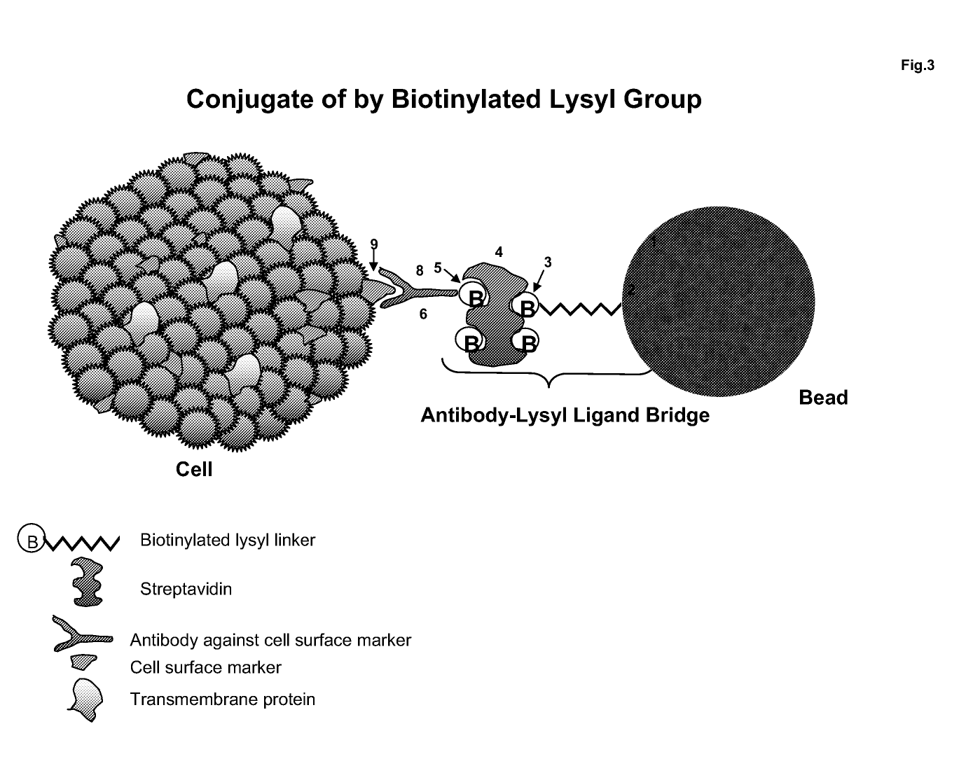

To prepare a robust, chemically flexible surface for the coupling of antibody, the present invention envisions use of the biotin-avidin complex or biotin-streptavidin, as a means of tethering antibody to the biodegradable particle surface. Referring to FIG. 3, the epsilon-NH₂ groups of lysyl of the copolymer are biotinylated using custom or commercially available biotinylation reagents. A suitable commercial reagent kit is Sigma product BK-101, which uses a sulfo-NHS biotinylation reagent. For some uses, a cleavable biotinylation reagent can be used as is found in, for example, the commercial kit BK-200 (Sigma). Upon incorporation of the biotin into the biodegradable polymer, separately prepared conjugates of antibody with avidin or streptavidin can be reacted with the biotinylated polymer. The avidin-antibody conjugates or alternatively streptavidin antibody conjugates can be prepared by standard methods using, for example, the cross-linking reagents listed above.

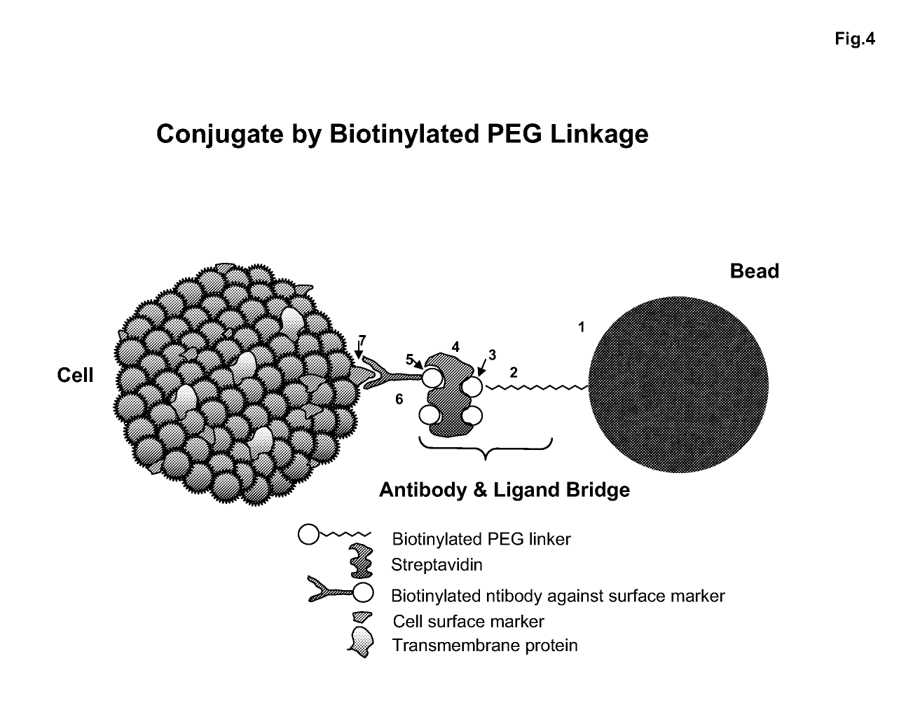

In an alternative embodiment the biodegradable polymer is covalently linked to avidin or streptavidin using cross-linking reagents such as carbodiimide, or other reagents as listed above. The avidin or streptavidin-linked biodegradable polymer is then reacted with biotinylated antibody to produce an antibody tethered, albeit noncovalently, to the biodegradable polymer particle. Referring to FIG. 4, these methods allow use of any biotinylated antibody to associate with the streptavidin surface, thus producing an antibody tethered to the surface that targets a cell surface marker.

Coupling of Antibodies By Antibody-Antibody Conjugation

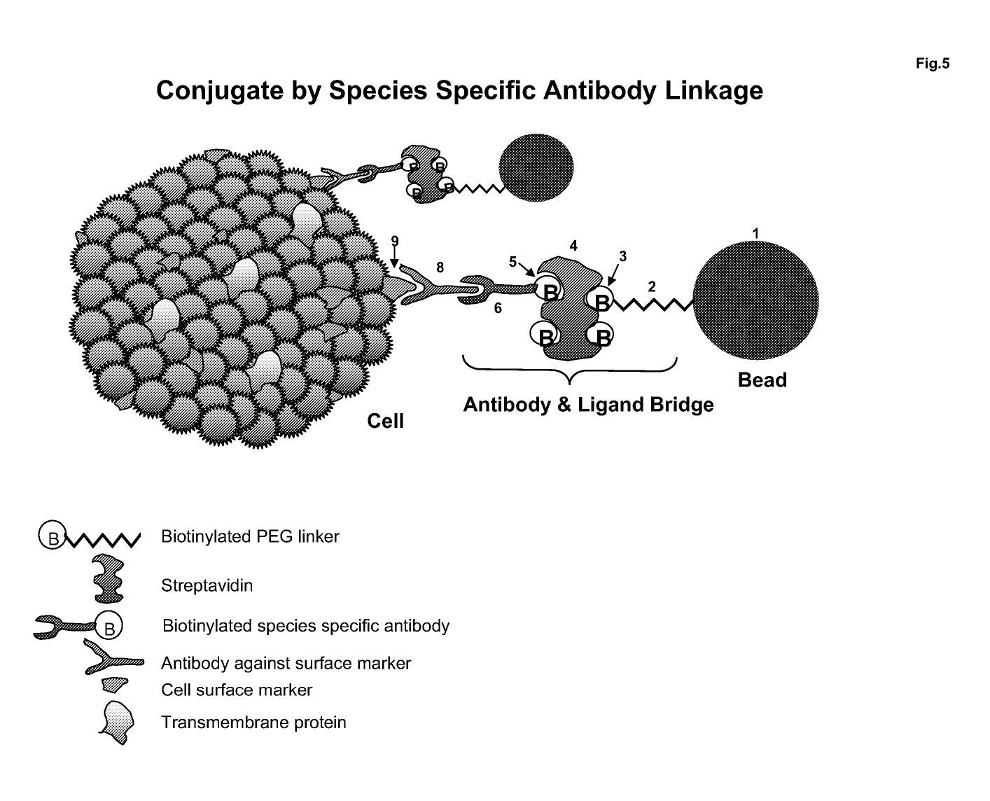

Referring now to FIG. 5, an alternative embodiment of the invention for antibody tethering is illustrated. FIG. 5 depicts use of a species-specific antibody directed against the F_{c} portion of the cell targeting antibody in an animal species different from the one used to raise antibody targeted to a cell surface marker. For example, an antibody against a cell surface marker in the mouse, is linked to an anti-F_{c} monoclonal antibody raised to the F_{c} marker of mice. The anti-F_{c} antibodies can be directly conjugated with the poly(lactic acid)—lysine copolymer or activated PEG linkage of the copolymer, thus creating an antibody surface targeting the respective cell surface markers. Alternatively, the species-specific antibodies can be biotinylated and then conjugated with the avidin or streptavidin surface on the polymer particles, as illustrated in FIG. 5. The present invention thus creates an antibody surface recognizing a group of antibodies sharing the common F_{c} domain. An advantage of this method is that the antibodies against the cell surface markers can be tethered onto the polymer particle surface without the need of prior chemical modification.

Selection of Antibodies Targeting Cell Surface Markers

In the present invention a wide range of antibodies to surface markers of hepatic cells and non-hepatic cells can be used. These antibodies include commercially available antibodies, antibodies prepared by the inventor, and antibodies prepared by others. These antibodies can include antibodies to ICAM-1, anti-ratRT1A^{a,b,1} or its human equivalent, anti-MHC I antibody, antibodies to integrins, antibodies to growth factor receptors, and antibodies to glycoproteins.

Examples of the Compositions and Uses of the Invention

The following specific examples are provided to better assist the reader in the various aspects of practicing the present invention. As these specific examples are merely illustrative, nothing in the following descriptions should be construed as limiting the invention in any way. Such limitations are of course, defined solely by the accompanying claims.

Use if the Biodegradable Polymer-Antibody Conjugates for Binding of Cells and Isolation of Cell Populations

The polymer particles tethered with antibody targeting cell surface markers are incubated with suspensions of a mixed population of cells under nearly physiological conditions. Thus temperatures between 0° and 40° C., pH between about 6 and about 7.5 and isotonic solutions are used. In one embodiment cells are incubated with particle-antibody conjugates at about 25° C., pH about 7.0 in Hank's BSS for about 30 minutes, or longer. The antibody-surface receptor interaction facilitates the binding of targeted cells to the polymer beads. The invention envisions interaction of multiple cells with each biodegradable polymer particle, or the interaction of several microparticle beads with a single cell, or any ratio there between. One skilled in the art can adjust the surface density of antibodies and the length of the tether to optimize interaction of cells and particles for any of multiple purposes. By these means a particular population of cells as identified by the antibody is attached to the particle-antibody conjugates. Thus, the particles permit a facile separation of one cell population from a mixed population. In other words, the present invention constitutes a positive sort method and enrichment of a select population of cells. The particle-antibody conjugates can equally well be used in a negative sort, or depletion procedure, that is, to eliminate cell populations considered not to be of interest by using antibodies selected for those particular populations.

In one particular example, the particle-antibody conjugates are used to isolate mesenchymal cells, to separate them from other cells including hepatic progenitors. The particle-antibody conjugates prepared with antibody to mesenchymal cells are incubated with a mixed cell population containing mesenchymal cells. After incubation the particles with adherent cells are isolated and seeded into a cell culture chamber with separate compartments. Other progenitor cells, for example, hepatic progenitors, are then seeded into other compartments. When, in this example, the compartments have a contiguous media connection, as, for example, in a Transwell® dish, then the remote interaction of hepatic progenitors and mesenchymal stem cells is observed.

The particles can be used to enrich a cell in a cell population by anchoring the cells to the particles. The cells anchored to the particles can be liver cells, hepatic precursors, fibroblasts, endocrine cells, endothelial cells, or any anchorage-dependent cell. The cells not anchored to the biodegradable particle can be any non-anchorage dependent cell including hemopoietic cells, hemopoietic precursors, erythrocytes, leukemic cells, and lymphoma cells, and cells that do not have the surface receptors targeted by the antibody-polymer surface.

Use of the Biodegradable Polymer-Antibody Conjugates For Ex Vivo Culture of Particle-Cell Conjugates and TheirUse In a Three-Dimensional Bioreactor

Biodegradable particles conjugated with extracellular matrix, as described above, are incubated with anchorage-dependent cells. The use of extracellular matrix provides a favorable growth environment for anchorage-dependent cells and permits facile transfer of cell suspensions from one container to another. Moreover, this method permits easy expansion of cell populations and easy sampling of cell populations.

Many varieties of anchorage-dependent cells are suitable for use with the biodegradable particle extracellular matrix conjugates including hepatic precursors, mesenchymal cells, mesenchymal precursors, muscle cells including cardiac cells, neuronal cells, glial cells, fibroblasts, stem cells, epithelial cells, and endothelial cells. Moreover, endocrine cells are also suitable for growth on particle-extracellular matrix conjugates.

The particle-cell combinations are also suitable for growth in three-dimensional culture in bioreactors. Such a use provides for flow of nutrient media and nutrient gases to an adherent cell population and ready exchange of metabolites and metabolic waste as necessary.

Use of the Biodegradable Polymer-Protein Conjugates For Cryopreservation of Anchorage-Dependent Cells

By attaching the enriched cells to a biodegradable polymer support, the composition of the present invention can also improve the survival and recovery of cryopreserved cells. Earlier methodologies for the cryopreservation of cells are successful for hemopoietic cells that normally exist in suspension, and for cell lines, that are adapted to cell culture, but work poorly for anchorage-dependent cell types. Cryopreservation of anchorage-dependent hepatocytes by the usual methods of resuspension using trypsin or other removal agents, leads to a very substantial loss in cell viability. Moreover, the cells lose their differentiated character and there is a loss of ability to attach to solid surfaces. The present invention applies derivatized biodegradable particles for anchorage of cells. The particle-extracellular matrix conjugates are provided for cell attachment, and then exposed to a vitrification solution, to prevent ice crystal formation. A suitable cryo-preservation or vitrification solution includes 5 to 15 percent, typically 10 percent, dimethyl sulfoxide (v/v) in serum supplemented medium. An alternative vitrification solution comprises ten percent (v/v) dimethyl sulfoxide in defined medium, that is, not containing serum or plasma. Moreover, the particle-bound cells do not have to be removed from the particles after thawing. This improvement is an important one, since cells embedded in alternative materials such as extracellular matrix, or alginate, must be resuspended after thawing to be of practical use for most research or clinical needs. Yet to enzymatically treat the cells immediately after thawing almost invariably results in loss of survival for the majority of cells. The cells are especially sensitive to handling and highly vulnerable to enzymatic treatments immediately after conventional cryopreservation and thawing. By avoiding the enzymatic treatment after thawing, the cells on the beads are much more robust. The cells on the particles can simply be rinsed with cell culture medium and used immediately without any further handling. This procedure improves the survival and function of cryopreserved anchorage-dependent cells and streamlines work of cell-banking and cell-typing.

Use of the Biodegradable Polymer-Protein Conjugates For Cell Transplantation

In yet another embodiment of the present invention, the methods of the invention provide a robust means for preparation of enriched anchorage-dependent cells for transplantation. Conjugates of biodegradable polymer-protein-cells are implanted directly into blood vessels or recipient organs. The polymer is designed to degrade into constituent molecules that are naturally present in vivo, in synergy with growth and maturation of the enriched progenitor cells and the formation of natural extracellular matrix and tissue structure. Moreover, the dissolution and clearance of the polymer materials is envisioned to minimize the problem of foreign body rejection.

Cell Enrichment By Negative Sorting

In cases where a desired cell type does not exhibit unique identifiable cell surface markers, a negative sort, optionally an iterative negative sort, can enrich the desired cell type in the population. An exemplary case follows.

A biodegradable particle-antibody to glycophorin A (particle-Ab(GA)) conjugate is prepared by the methods described above. A substantially single cell suspension of 10⁷ embryonic liver cells at a concentration of 10⁶ cells/ml is mixed with 0.5 g wet weight of particle-Ab(GA) conjugate. By “substantially” in this context is meant that at least about 70% of the cells are unassociated with other cells. In one embodiment, a substantially single cell suspension has at least about 90% of the cells unassociated with other cells. The mixture is incubated at 24° C. for one hour in defined medium (HDM) consisting of a 1:1 mixture of Dulbecco's modified Eagle's medium and Ham's F12 (DMEM/F12, GIBCO/BRL, Grand Island, N.Y.), to which is added 20 ng/ml EGF (Collaborative Biomedical Products), 5 μg/ml insulin (Sigma), 10⁻⁷M Dexamethasone (Sigma), 10 82 g/ml iron-saturated transferrin (Sigma), 4.4×10⁻³M nicotinamide (Sigma), 0.2% (w/v) Bovine Serum Albumin (Sigma), 5×10⁻⁵M 2-mercaptoethanol (Sigma), 7.6 μeq/1 free fatty acid, 2×10⁻³M glutamine (GIBCO/BRL), 1×10⁻⁶M CuSO₄, 3×10⁻⁸M H2SeO₃ and antibiotics. The cells remaining in the supernatant and not attached to the beads are cultured in fresh medium or subjected to a subsequent sorting.

Cell Enrichment By Positive Sorting

In cases where a desired cell type exhibits at least one unique identifiable cell surface marker, a positive sort, optionally an iterative positive sort or a combination of a positive and negative sort, can enrich for the desired cell type in the population. An exemplary case follows.

A biodegradable particle-antibody to ICAM-1 (particle-Ab (ICAM-1)) conjugate is prepared by the methods described above. A single cell suspension of 10⁷ embryonic liver cells at a concentration of 10⁶ cells/ml is mixed with 0.5 g wet weight of particle-Ab(ICAM-1) conjugate. The mixture is incubated at 24° C. for one hour in defined medium (HDM) consisting of a 1:1 mixture of Dulbecco's modified Eagle's medium and Ham's F12 (DMEM/F12, GIBCO/BRL, Grand Island, N.Y.), to which is added 20 ng/ml EGF (Collaborative Biomedical Products), 5 μg/ml insulin (Sigma), 10⁻⁷M Dexamethasone (Sigma), 10 μg/ml iron-saturated transferrin (Sigma), 4.4×10⁻³M nicotinamide (Sigma), 0.2% (w/v) Bovine Serum Albumin (Sigma), 5×10⁻⁵M 2-mercaptoethanol (Sigma), 7.6 [eq/1 free fatty acid, 2×10⁻³M glutamine (GIBCO/BRL), 1×10⁻⁶M CuSO₄, 3×10⁻⁸MH₂ SeO₃ and antibiotics. The cells attached to the particles are cultured in fresh medium.

In another example, a biodegradable particle-antibody to EpCAM-1 (particle-Ab (EpCAM-1))/NCAM-1 (particle-Ab (NCAM-1)) conjugate is prepared by the methods described above. In yet another embodiment, a biodegradable particle-antibody to EpCAM-1 (particle-Ab (EpCAM-1))/ICAM-1 (particle-Ab (ICAM-1)) conjugate is prepared by the methods described above. Such biodegradable particle-antibody with at least one unique identifiable cell surface marker can be used to enrich for the desired cell type in the population.

Cell Culture On Particle-ECM Conjugates

A population of hepatic progenitor cells enriched by any method is incubated with biodegradable particles conjugated with collagen IV in HDM. Collagen IV-particles are prepared by the methods above to yield 500 micron diameter particles with a collagen IV to particle ratio of 0.02 (w/w). Ten grams total wet weight of collagen IV-particles are suspended in 500 ml of HDM at 37° C., with a 95% (v/v) air/ 5% (v/v) CO₂ atmosphere. The collagen IV-particles are seeded with 10⁶ hepatic progenitors and the medium changed every second day. The particles are kept suspended by gentle agitation. The culture is monitored for cell metabolism by changes in pH and glucose concentration and for cell growth by determining the DNA content. New growing surfaces are provided for growing cultures by adding fresh particles to the culture mixture.

In yet other examples, a population of hepatic progenitor cells enriched by any method is incubated with biodegradable particles conjugated with other any other suitable specialized matrix chemistry generally present in, without limitation, fetal forms of laminin, hyaluronic acid, and heparin glycan sulphate as known to those of skill in the art.

Cell Cryopreservation Using Particle-Adherent Cells

Anchorage-dependent cells growing on biodegradable particles, as in example 6.4, are cryopreserved by resuspending the particles with adherent cells in a solution of 10% (v/v) dimethyl sulfoxide in HDM and transferring an aliquot containing about 1×10⁶ cells to a sterile ampoule or vial. The ampoule or vial is appropriately sealed and the temperature gradually reduced at about 1° C. per minute to between about −80° C. and about −160° C. The cells are stored at about −160° C. indefinitely until needed. When needed, an ampoule or vial is rapidly thawed, as for example in a tepid water bath. The contents are then aseptically transferred to a culture vessel with culture medium, HDM.

Transplantation of Hepatic Progenitors In a Model Of Liver Failure

A rat model of liver failure is used to evaluate heterogenous cell transplantation therapy. Liver failure is modeled by surgical removal of about 70% of the liver and/or ligation of the common bile duct in an experimental group often male rats (125 to 160 g body weight). A sham control group of ten age- and sex-matched rats is subjected to a similar anesthesia, mid-line laparotomy, and manipulation of the liver, but without ligation of the bile ducts and without hepatectomy.

An enriched population of hepatic precursors anchored to biodegradable beads is prepared as described above. In brief, the livers of 12 embryonic (embryonic day 14) rat pups are aseptically removed, diced, rinsed in 1 mM EDTA in Hank's BSS without calcium or magnesium, pH 7.0, then incubated for up to 20 minutes in Hank's BSS containing 0.5 mg/ml collagenase to produce a near single cell suspension.

Aseptic biodegradable particles conjugated with antibody to ICAM-1 are prepared as above. The single cell liver suspension from twelve pups is incubated with 1.5 ml of packed volume of ICAM-1-microparticles for one hour at 25° C. The particles are then diluted in ten volumes of HDM and decanted after standing at 1×g for five minutes. The procedure is then repeated. The particles are gently resuspended in fresh HDM and incubated at 37° C, in an atmosphere of 95% air, 5% CO₂ (v/v) for five days.

On day three after the hepatectomy or sham operation, the rats, both experimental and sham control, are subjected to a 5 mm abdominal incision to expose the spleen. One half of each of the experimental and sham control group animals, randomly chosen, are injected with 0.1 ml each of the biodegradable-particle-ICAM-1-embryonic liver cell composition, directly into the spleen. All incisions are closed with surgical staples. The immunosuppressant cyclosporine A, 1 mg/kg body weight, is administered daily intraperitoneally.

Blood levels of bilirubin, gamma glutamyl transferase and alanine aminotranferase activities are monitored two days before the hepatectomy or sham hepatectomy operation and on post-operation days 3, 7, 14, and 28. Body weight, water consumption, and a visual inspection of lethargy are recorded on the same days. At 28 days post hepatectomy all surviving animals are killed for histological evaluation of spleen and liver.

All publications, patents, and patent documents referred to herein are hereby incorporated in their respective entireties by reference.

The invention has been described with reference to the foregoing specific and preferred embodiments and methods. However, it should be understood that many variations may be made while remaining within the spirit and scope of the invention. Therefore, the foregoing examples are not limiting, and the scope of the invention is intended to be limited only by the following claims.

TABLE 1
PREPARATION OF FREE FATTY ACID (FFA) MIXTURE
Source of Purified Fatty Acids: See Table 2
Preparation of the stocks The free fatty acids are prepared by dissolving
each individual component in 100% ethanol. Comments are as follows:

| | |
|---|---|
| Palmitic acid (solid) | 1 M stock; soluble in hot alcohol |
| Palmitoleic acid | 1 M stock; readily soluble in alcohol |
| Oleic acid | 1 M stock; readily soluble in alcohol |
| Linoleic acid | 1 M stock; readily soluble in alcohol |
| Linolenic acid | 1 M stock; readily soluble in acohol |
| Stearic acid (solid) | 151 mM stock, soluble in alcohol at 1 gram in |
| | 21 mls and must be heated. |

These stocks can be stabilized by bubbling nitrogen through each of them
and then storing them at −20° C.

The free fatty acid mixture stock solution:

| | |
|---|---|
| Palmitic acid | 31.0 mM |
| Palmitoleic acid | 2.8 mM |
| Oleic acid | 13.4 mM |
| Linoleic acid | 35.6 mM |
| Linolenic acid | 5.6 mM |
| Stearic acid | 11.6 mM |

This yields a combined total of 100 mM free fatty acids. This stock with
all the free fatty acids can be stabilized also by bubbling through nitrogen
and then storing it at −20° C.
Final Solution:
Add 76 μL of the free fatty acid mixture stock per liter of culture medium
to achieve a final concentration of 7.6 μEq. The free fatty acids are toxic
unless they are presented with purified, fatty acid-free, endotoxin-free
serum albumin (e.g. Pentex type V albumin). Albumin is prepared in the
basal medium or PBS to be used and at a typical concentration of
0.1-0.2%.

TABLE 2
Sources of Basal Media, Growth Factors, Matrix Components
and other Culture Components

| | |
|---|---|
| FACTORS | VENDOR(S) |

Growth Factors/Hormones

| | |
|---|---|
| Prolactin (Luteotropic Hormone) | Sigma-Aldrich |
| | US Biological |
| | Cortex Biochemicals Inc. |
| | ICN Biomedicals |
| Epidermal Growth Factor (EGF) | |
| Mouse; receptor grade | Collaborative Biomedicals |
| Human recombinant | Sigma-Aldrich |
| | Pepro Tech |
| | Upstate Biologicals |
| | Accurate Chemicals |
| | Clonetics Products |
| | Antigenix America Inc. |
| Mouse recombinant | Accurate Chemicals |
| | Antigenix America Inc. |
| Transferrin: holo-Iron Saturated | Sigma-Aldrich |
| Bovine, human | Clonetics |
| Somatotropin: Growth Hormone | |
| Human Pituitary | Sigma-Aldrich |
| Human Recombinant | Accurate Chemicals |
| | ICN Biomedicals |
| Hydrocortisone | Sigma-Aldrich |
| | Clonetics |
| | Calbiochem |
| | Alfa Aesar |
| | Bishop Canada |
| | ICN Biomedicals |
| Dexamethasone | Sigma-Aldrich |
| | Clonectics |
| | Amersham Pharmacia Biotech |
| | Accurate Chemicals |
| | Calbiochem |
| | ICN Biomedicals |
| Glucagon | Sigma-Aldrich |
| Porcine Pancreas | BIOTREND Chemikalien |

OTHER SUPPLEMENTS

| | |
|---|---|
| HDL: High Density Lipoprotein | |
| Human plasma | Sigma-Aldrich |
| | Chemicon International |
| | Biodesign International |
| | Per Immune |
| | BioResource Technology |
| | Academy Biomedical Co. |
| | Biodesign International |
| Free Fatty Acids | |
| Linoleic | Sigma-Aldrich |
| | Altech Associates Inc., |
| | ICN Biomedicals |
| Linolenic | Sigma-Aldrich |
| | Altech Associates Inc. |
| Oleic | Sigma-Aldrich |
| | Altech Associates Inc., |
| | ICN Biomedicals |
| Palmitic | Sigma-Aldrich |
| | Altech Associates Inc., |
| | ICN Biomedicals |
| Stearic | Sigma-Aldrich |
| | Altech Associates Inc., |
| | ICN Biomedicals |
| Bovine Serum Albumin V | Sigma-Aldrich |
| Fatty Acid Free | Genmini Bio-Products |
| Nicotinamide (Niacinamide) | Sigma |
| | Calbiochem |
| | ICN Biomedicals |
| | Spectrum Laboratory Products |
| | TCI America |
| Putrescine | Sigma-Aldrich |
| | Advanced ChemTech Inc. |
| | Crescent Chemicals |
| | ICN Biomedicals |
| | Spectrum Laboratory Products |
| 3′,3′,5′-Triiodo-L-thyronine (T3) | Sigma-Adlrich |
| | Toronto Research Chemicals |
| | ICN Biomedicals |
| | Novabiochem |
| | TCI America |

TRACE ELEMENTS

| | |
|---|---|
| Copper Pentahydrate | Sigma-Aldrich |
| | Chem Services Inc. |
| | Crescent Chemicals |
| | Gallade Chemical, Inc. |
| | ICN Biomedicals |
| | MV Laboratories, Inc. |
| | Specturm Laboratory Products |
| | Strem Chemicals, Inc. |
| Zinc Sulfate Heptahydrate | Sigma-Aldrich |
| | Crescent Chemicals |
| | ICN Biomedicals |
| | MV Laboratories, Inc. |
| Selenious Acid: | Sigma-Aldrich |
| | ICN Biomedicals |
| | MV Laboratories |
| | Spectrum Laboratory Products |

BASAL MEDIA

| | |
|---|---|
| DMEM/F12 | Gibco BRL |
| | BioWhittaker |
| | Mediatech Inc. |
| | Specialty Media-Division |
| | of Cell & Molecular |
| | Technologies |
| RPMI 1640 | Gibco BRL |
| | Biologos Inc. |
| | BioSource International |
| | ICN Biomedicals |
| | BioWhittaker |
| Hepatocyte Medium | Sigma, Clonetics |
| Keratinocyte Basal Medium | Clonetics |

Extracellular Matrix Components

| | |
|---|---|
| Fibronectin | |
| Bovine | Sigma-Aldrich |
| | Collaborative Biomedical |
| | Accurate Chemicals |
| Human | Biosource International |
| Bovine, Human, Rat, Mouse | BIOTREND Chemikalien |
| Human | Chemicon International |
| Bovine, Chicken, Horse, Human, Mouse, | Calbiochem |
| Bovine, Human, Mouse | |
| Salmon, Rat | |
| Laminin | |
| Mouse | Sigma-Aldrich |
| | Collaborative Biomedical |
| | EY Laboratories |
| | Alexis Corp. |
| Human | BioSource International |
| | Alexis Corp. |
| | Chemicon International |
| | BIOTREND Chemikalien |
| Collagen Type I | Collaborative Biomedical |
| | Sigma-Aldrich |
| | BioShop Canada |
| | BIOTREND Chemikalien |
| Collagen Type II | Sigma-Aldrich |
| | Chemicon International, Inc. |
| | Accurate Chemicals |
| Collagen Type III | Chemicon International, Inc. |
| | Accurate Chemicals |
| | BIOTREND Chemikalien |
| Collagen Type IV | Collaborative Biomedical |
| | Sigma-Aldrich |
| | BIOTREND Chemikalien |
| Matrigel | Collaborative Biomedical |
| | Clonetics |
| Unbleached heparins | Sigma |
| | BioChemika |
| | Clonetics |
| | CarboMer, Inc. |
| | Alfa Aesar |
| | PolySciences, Inc |
| Heparan sulfates | Sigma-Aldrich |
| | BioChemika |
| | CarbMer, Inc. |
| | US Biologicals |
| | Seikagaku USA |
| | Calbiochem |
| | ICN Biomedicals |
| Carrageenans (heparin-like reagents | Sigma-Aldrich |
| purified from seaweed. There are | BioChemika |
| three forms available: lamda, | CarboMer, Inc. |
| kappa and iota that vary | ICN Biomedicals |
| in their solubility) | TCI America |
| Suramin (heparin-like molecule found to | Sigma-Aldrich |
| have potent anti-microbial activity | BioChemika |
| and anti-tumor activity) | Calbiochem |
| | Alexis Corp. |
| | BIOMOL Research |
| | Laboratories, Inc. |
| | ICN Biomedicals |
| | A.G. Scientifics |
| | American Qualex |
| | International Inc. |
| Heparan sulfate proteoglycan (HS-PG) | Collaborative Biomedical |
| from EHS tumor | Sigma- |
| | Aldrich |
| | Chemicon International |

What is claimed is:

1. A method of cryopreservation of anchorage-dependent cells comprising
(a) allowing the cells to anchor to a composition comprising at least one biodegradable particle to form a mixture, and
(b) freezing the mixture.
(c) thawing and recovery of cells from the cells-polymer particle conjugates.

2. The method of claim 1, wherein the biodegradable particle further comprises a receptive group covalently linked to the particle.

3. The method of claim 2, wherein the receptive group comprises an antibody, a fragment of an antibody, an avidin, a streptavidin, a biotin moiety, or combinations thereof.

4. The method of claim 1 further comprising an extracellular matrix.

5. The method of claim 1 further comprising a cryopreservation solution.

6. The method of claim 5, wherein the cryopreservation solution comprises 10 % (v/v) dimethyl sulfoxide.

7. A method of separating cells comprising:
(a) providing a composition comprising at least one biodegradable particle, at least one receptive group covalently linked thereto, at least one cell anchored to at least one receptive group, and at least one cell not anchored thereto, and
(b) removing at least one cell not anchored to the biodegradable particle.

8. The method of claim 7, wherein the receptive group is an antibody, a fragment of an antibody, an avidin, a streptavidin, a biotin moiety, or combinations thereof.

9. The method of claim 7, wherein the cell anchored to the biodegradable particle comprises a liver cell or a hepatic precursor.

10. The method of claim 7, wherein the cell not anchored to the biodegradable particle comprises a hemopoietic precursor.

11. A method of cell culture of anchorage-dependent cells comprising
(a) providing a composition comprising at least one biodegradable particle, at least one receptive group covalently linked thereto, and at least one cell adherent to said at least one receptive group; and
(b) contacting the composition with cell culture medium.

12. The method of claim 1 1, wherein the composition further comprises extracellular matrix.

13. The method of claim 11, wherein the cell comprises at least one of a hepatic precursor, a hemopoietic precursor, a fibroblast, a mesenchymal cell, a cardiac cell, an endothelial cell, an epithelial cell, a neuronal cell, a glial cell, an endocrine cell, or combinations thereof.

14. A treatment of a subject in need of cell therapy, comprising administering to the subject an effective amount of a composition comprising at least one biodegradable particle, at least one receptive group covalently linked thereto, and at least one cell anchored to said at least one receptive group.

15. The treatment of claim 14, wherein the cell comprises a hepatic progenitor.

16. The treatment of claim 14, wherein the composition is administered intravenously, intra-arterially, intramuscularly, parenterally, or in any combination thereof.

17. The treatment of claim 14, wherein the effective amount falls in the range of from about 10² to about 10¹¹ cells.
